(19) United States
(12) Patent Application Publication
(10) Pub. No.: US 2015/0320849 A1
(43) Pub. Date: Nov. 12, 2015

(54) Method of treating central nervous system disorders with Borrelia burgdorferi antigen

(71) Applicant: BEECH TREE LABS, INC., Delanson, NY (US)

(72) Inventor: John McMichael, Delanson, NY (US)

(21) Appl. No.: 14/704,084

(22) Filed: May 5, 2015

Related U.S. Application Data
(60) Provisional application No. 62/007,715, filed on Jun. 4, 2014.
(60) Provisional application No. 61/989,222, filed on May 6, 2014.

(51) Int. Cl. A61K 39/02

(52) U.S. Cl. CPC A61K 39/0225; A61K 2039/521

(57) ABSTRACT

The present invention relates to a treatment for mammalian subjects suffering from a central nervous system (CNS) disorder by administering a composition comprising an Borrelia burgdorferi antigen at a sub-vaccine level effective to alleviate symptoms of the CNS disorder.

### CROSS-REFERENCE TO RELATED APPLICATIONS

This application claims the benefit of priority to U.S. Provisional Patent Application No. 61/989,222 filed May 6, 2014 and U.S. Provisional Patent Application No. 62/007,715 filed Jun. 4, 2014, the disclosures of which are incorporated herein by reference in their entireties.

### BACKGROUND OF THE INVENTION

A vaccine comprising a recombinant B. burgdorferi outer-surface lipoproteins (OspA) has been tested in animals and in humans. This Lyme disease vaccine (LYMErix™, SmithKline Beecham Biologicals) has been shown to serve as a protective antigen in animals and as a safe immunogen in humans. The LYMErix™ vaccine is administered at a dosage of 30 micrograms of recombinant OspA lipoprotein at 0, 1 and 12 months with all three doses required to achieve optimal protection. B. burgdorferi expresses OspA while residing in the midgut of the infected tick, but OspA is down regulated after tick attachment and is usually undetectable or absent when B. burgdorferi is inoculated into the human host. Schwan et al. Proc. Natl. Acad. Sci. (USA) 92:2909-2913 (1995). Therefore, a novel hypothesis has been proposed to explain the effectiveness of lipoprotein OspA vaccination. When infected ticks bite humans who have been vaccinated with recombinant OspA, the vaccine-induced antibodies are taken up by the tick and interact with the B. burgdorferi in the midgut of the tick, thereby preventing transmission of the organism to the host. This mechanism has been suggested by a pre-clinical study in which B. burgdorferi were detected by immunofluorescence assay in none of the ticks that fed on OspA-immunized mice, compared with 72% of ticks that fed on control-immunized mice. Fikrig et al. Proc. Natl. Acad. Sci. (USA) 89:5418-5421 (1992).

Of interest to the present application is the disclosure of Matman in Cell Wall Deficient Forms-Stealth Pathogens (3^{rd} Edition) of cases of amyotrophic lateral sclerosis (ALS) in which drawn blood yielded a spirochete which reacted against of Borrelia burgdorferi antibody.

The disclosure of co-owned U.S. Pat. No. 6,592,875 is hereby incorporated by reference in its entirety. U.S. Pat. No. 6,592,875 discloses the use of Borrelia burgdorferi antigen at a sub-vaccine level is effective to alleviate the symptoms of Lyme disease. U.S. Pat. No. 6,592,875 does not disclose or suggest the use of Borrelia burgdorferi antigen at a sub-vaccine level to alleviate the symptoms of Parkinson's Disease and ALS.

### SUMMARY OF THE INVENTION

The present application relates to the discovery that administration of Borrelia burgdorferi antigen(s) at a sub-vaccine level is effective to alleviate symptoms of central nervous system (CNS) disorders such as Parkinson's disease or amyotrophic lateral sclerosis (ALS).

The invention provides methods for treating a mammalian subject suffering from a central nervous system (CNS) disorder comprising administering a composition comprising a Borrelia burgdorferi antigen at a sub-vaccine level effective to alleviate symptoms of the CNS disorder. In some embodiments, the CNS disorder is selected from the group consisting of Parkinson's Disease and amyotrophic lateral sclerosis (ALS).

Suitable antigens for use according to the invention include outer surface lipoproteins of Borrelia burgdorferi such as the outer surface lipoproteins OspA, OspB, OspC and the antigen LFA-a. In some embodiments, the antigen is a lysed Borrelia burgdorferi antigen produced by treatment of the organism in phenol followed by multiple freeze/thaw cycles. Borrelia burgdorferi for use according to the invention is publically available as ATCC deposit 35210.

The Borrelia burgdorferi antigens described herein are administered at sub-vaccine levels. “Sub-vaccine levels” are defined as levels less than those sufficient to induce an effective humoral immune response as exemplified by the determination of a positive wheal upon subcutaneous injection. In some embodiments, methods described herein comprise administering a composition comprising 2×10⁻⁷ g to about 1×10⁻¹² g of the outer surface lipoprotein per dose. In some embodiments, from one to four doses of the composition are administered daily. The compositions described herein are administered to a subject by a method selected from the group consisting of sublingual, subcutaneous, intravenous, intramuscular, and intrathecal administration. In some embodiments, the compositions described herein are administered by sublingual administration or subcutaneous administration.

In some embodiments, the composition is administered to the subject in a single dose of about 0.05 cc in a pharmaceutically acceptable carrier, excipient or diluent.

Also described herein are compositions comprising (a) Borrelia burgdorferi antigens at a sub-vaccine level effective to alleviate symptoms of a central nervous system (CNS) disorder and (b) a pharmaceutically acceptable carrier. In some embodiments, the composition comprises from about 2×10⁻⁷ g to about 1×10⁻¹² g of the Borrelia burgdorferi per dose. In some embodiments, the composition comprises about 1 ng of the Borrelia burgdorferi per dose.

Additional aspects, features and variations of the invention will be apparent from the entirety of this application, including the detailed description, and all such features are intended as aspects of the invention. It should be understood, however, that the detailed description and the specific examples are given by way of illustration, and that the many various changes and modifications that will be apparent to those familiar with the field of the invention are also part of the invention.

Aspects of the invention described with the term “comprising” should be understood to include the elements explicitly listed, and optionally, additional elements. Aspects of the invention described with “a” or “an” should be understood to include “one or more” unless the context clearly requires a narrower meaning.

### DETAILED DESCRIPTION OF THE INVENTION

The present application relates to the discovery that the administration of Borrelia burgdorferi antigens at a sub-vaccine levels is effective to alleviate symptoms of central nervous system (CNS) disorders such as Parkinson's disease or amyotrophic lateral sclerosis (ALS).

The dose of Borrelia burgdorferi antigen to be administered to the subject is a sub-vaccine level (2-10 times a five-fold dilution of a vaccine level (which vaccine level is typically about 30 μg)) and can be determined by the physician, taking into account, age, sex, weight, etc. of the subject. In some embodiments, the administered dose of Borrelia burgdorferi antigen ranges from about 1×10⁻¹² g to about 2×10⁻⁷ g. In some embodiments, Borrelia burgdorferi antigen is administered 1, 2, 3, 4, 5, 6, 7, 8, 9, 10 or more times daily for a period of 1, 2, 3, 4, 5, 6 or more weeks. Additional therapy may be administered on a period basis, for example, daily, weekly or monthly. When the composition is administered sublingually, it is preferred that four to six doses be administered daily.

The subjects treated in the methods disclosed herein in its many embodiments are desirably human subjects, although it is to be understood that the principles of the presently disclosed subject matter indicate that the presently disclosed subject matter is effective with respect to invertebrate and to all vertebrate species, including mammals, which are intended to be included in the term “subject.”

In some embodiments, the methods disclosed herein will result in a slowing of the neurodegenerative disorder progression. As used herein, “slowing neurodegenerative disorder progression” means the delay of a clinically undesirable change in one or more disabilities in an individual suffering from a neurodegenerative disorder, such as ALS, and is assessed by methods routinely practiced in the art, for example, the revised Amyotrophic Lateral Sclerosis Functional Rating Scale (ALSFRS), pulmonary function tests, and muscle strength measurements. Such methods are herein referred to as “indicators of ALS disease progression.” Such delay can be shown in a controlled study comparing the rate of progression of disease in treated subjects versus untreated controls. Slowing progression alternatively means the lessening of the severity of a clinically undesirable changes as assessed at a particular point in time, compared to untreated controls.

An “improvement in a indicator of neurodegenerative disorder progression” as used herein refers to slowing of the rate of change in one or more of the indicators of a neurodegenerative disorder progression, such as ALS disease progression. An improvement in an indicator of ALS disease progression also includes a lack of a measurable change in one or more of the indicators of ALS disease progression. An improvement in an indicator of ALS disease progression additionally includes a positive change in one of the indicators of ALS disease progression described herein, such as, for example, an increase in an ALSFRS-R score. It is within the abilities of a physician to identify a slowing of disease progression in an individual suffering from ALS, using one or more of the disease assessment tests described herein. A physician may administer to the individual diagnostic tests, such as additional pulmonary function tests or muscle strength measurement tests, to assess the rate of disease progression in an individual suffering from ALS.

A slowing of neurodegenerative disorder progression may further comprise an “increase in survival time” in an individual suffering from the neurodegenerative disorder, e.g., ALS. A physician can use one or more of the disease assessment tests described herein to predict an approximate survival time of an individual suffering from ALS. A physician may additionally use the known disease course of ALS accompanied by a particular ALS mutation to predict survival time.

The “revised ALS functional rating scale” or “ALSFRS-R” is used by physicians and is a validated rating instrument for monitoring the progression of disability in ALS patients. The ALSFRS-R includes 12 questions that ask a physician to rate his or her impression of an ALS patient's level of functional impairment in performing one of ten common tasks, for example, climbing stairs. Each task is rated on a five-point scale, where a score of zero indicates an inability to perform a task and a score of four indicates normal ability in performing a task. Individual item scores are summed to produce a reported score of between zero (worst) and 48 (best).

The Borrelia burgdorferi antigen(s) described herein may be formulated in pharmaceutical compositions with a pharmaceutically acceptable excipient, carrier, or diluent.

In some embodiments, the Borrelia burgdorferi antigen(s) are formulated with pharmaceutically acceptable excipients such as carriers, solvents, stabilizers, adjuvants, diluents, etc., depending upon the particular mode of administration and dosage form. The pharmaceutical compositions should generally be formulated to achieve a physiologically compatible pH, and may range from a pH of about 3 to a pH of about 11, preferably about pH 3 to about pH 7, depending on the formulation and route of administration. In alternative embodiments, it may be preferred that the pH is adjusted to a range from about pH 5.0 to about pH 8. More particularly, the pharmaceutical compositions comprises in various aspects a therapeutically effective amount of at least one composition as described herein, together with one or more pharmaceutically acceptable excipients.

The term “pharmaceutically acceptable excipient” refers to an excipient for administration of a pharmaceutical agent, such as the compounds described herein. The term refers to any pharmaceutical excipient that may be administered without undue toxicity.

Pharmaceutically acceptable excipients are determined in part by the particular composition being administered, as well as by the particular method used to administer the composition. Accordingly, there exists a wide variety of suitable formulations of pharmaceutical compositions (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants (e.g., ascorbic acid), chelating agents (e.g., EDTA), carbohydrates (e.g., dextrin, hydroxyalkylcellulose, and/or hydroxyalkylmethylcellulose), stearic acid, liquids (e.g., oils, water, saline, glycerol and/or ethanol) wetting or emulsifying agents, pH buffering substances, and the like. Liposomes are also included within the definition of pharmaceutically acceptable excipients.

Additionally, the pharmaceutical compositions may be in the form of a sterile injectable preparation, such as a sterile injectable aqueous emulsion or oleaginous suspension. This emulsion or suspension may be formulated by a person of ordinary skill in the art using suitable dispersing or wetting agents and suspending agents. The sterile injectable preparation may also be a sterile injectable solution or suspension in a non-toxic parenterally acceptable diluent or solvent, such as a solution in 1,2-propane-diol.

Among the acceptable vehicles and solvents that may be employed are water, Ringer's solution, and isotonic sodium chloride solution. In addition, sterile fixed oils may be employed as a solvent or suspending medium. For this purpose any bland fixed oil may be employed including synthetic mono- or diglycerides. In addition, fatty acids (e.g., oleic acid) may likewise be used in the preparation of injectables.

Efficacy of treatment of symptoms associated with Parkinson's disease or ALS with Borrelia burgdorferi antigen can be assessed by methods known in the art. For example, one of ordinary skill in the art could determine the disease status of the of subjects suffering from Parkinson's disease and ALS by use of various assessments such as the Sf-36, PDQ-8, PDQ-39, 6-minute walk, handgrip dynamometer, vision acuity, speech acuity tests and the like.

The following Examples illustrate exemplary embodiments of the invention and provide evidence of the effectiveness of claimed treatment methods and compositions. Numerous improvements and further aspects of the invention are apparent to the skilled artisan upon consideration of the Examples which follow.

### EXAMPLE 1

A 59-year-old female has been diagnosed with ALS. Symptoms included difficulty standing, breathing and hoarseness in her voice. After initial administration of the Borrelia burgdorferi antigen, the subject reported that she “felt different, could breathe deeper, could lift legs more easily, stand up more easily, and walking was better.”

The subject was treated with sublingual administration two to four times daily of one drop (0.05 mL) a composition comprising a dilution of a commercially available recombinant OspA Lyme disease vaccine (Boehringer Ingelheim). The diluted composition comprises about 0.25×10⁻⁶ g of recombinant lipoprotein OspA adsorbed onto aluminum as aluminum hydroxide adjuvant in phosphate buffered saline further comprising 2-phenoxyethanol as a bacteriostatic. After a week of treatment, the subject reported that her legs felt stronger and that she was walking better. The subject's clinician noted that the subject's voice sounded stronger.

After three weeks of treatment, the subject reported that speech was improved, and that she could walk farther without shortness of breath. Two days later, the subject reported that her legs were not twitching, not hot and cold as they had been. The subject also reported that she was able to exercise. After an additional two days of treatment, the subject reported that she could cross her legs without using her arms to lift her legs. The subject continues the treatment at one drop, four to five times daily.

### EXAMPLE 2

A 76-year-old male subject has been diagnosed with Parkinson's disease. After nine days of taking one drop of a Borrelia burgdorferi antigen composition described herein four times daily, the subject reported a decrease in tremors.

### EXAMPLE 3

A 72-year-old female subject has been diagnosed with Parkinson's disease. Complications of disease led to a fall resulting in a broken hip, surgery and several months of rehab.

After two months of drop therapy (one drop, four times daily), the subject noticed no improvement and discontinued the therapy. Within two weeks of stopping the drops, the subject noticed decline relative to the Parkinson's symptoms. The drop therapy was resumed and subject notice an increased stability when walking or standing. Subject continues the treatment at one drop, four to five times daily.

Numerous modifications and variations in the practice of the invention are expected to occur to those skilled in the art upon consideration of the presently preferred embodiments thereof. Consequently, the only limitations which should be placed upon the scope of the invention are those which appear in the appended claims.

What is claimed is:

1. A method of treating a mammalian subject suffering from a central nervous system (CNS) disorders comprising administering a composition comprising an Borrelia burgdorferi antigen at a sub-vaccine level effective to alleviate symptoms of the CNS disorder.

2. The method of claim 1, wherein the CNS disorder is Parkinson's Disease and amyotrophic lateral sclerosis (ALS).

3. The method of claim 1, wherein the antigen is an outer surface lipoprotein of Borrelia burgdorferi.

4. The method of claim 3, wherein the outer surface lipoprotein is OspA.

5. The method of claim, 1 wherein lysed Borrelia burgdorferi is administered as the antigen.

6. The method of claim 1, wherein said composition comprises from about 1×10⁻¹² g to about 2×10⁻⁷ g of Borrelia burgdorferi outer surface lipoprotein per dose.

6. The method of claim 5 wherein said composition comprises about 1×10⁻⁹ g of the outer surface lipoprotein per dose.

7. The method of claim 1, wherein the composition is administered to the subject multiple times daily.

8. The method of claim 1 where the composition is administered to the patient by method selected from the group consisting of sublingual, subcutaneous, intravenous, intramuscular and intrathecal administration.

9. The method of claim 8, wherein the composition is administered to the subject by sublingual administration.

10. A composition for treatment of the symptoms of a central nervous system (CNS) disorder comprising (a) a Borrelia burgdorferi antigen at a sub-vaccine level effective to alleviate symptoms of the CNS disorder and (b) a pharmaceutically-acceptable carrier, diluent or adjuvant.

11. The composition of claim 10, wherein the Borrelia burgdorferi antigen is an outer surface lipoprotein.

12. The composition of claim 11, wherein the outer surface lipoprotein is OspA.

13. The composition of claim 10 wherein lysed Borrelia burgdorferi is the antigen.

14. composition of claim 10, comprising from about 1×10⁻¹² g to about 2×10⁻⁷ g of Borrelia burgdorferi outer surface lipoprotein per dose.
